(19) United States
(12) Patent Application Publication
(10) Pub. No.: US 2016/0228648 A1
(43) Pub. Date: Aug. 11, 2016

(54) SHOULDER ADAPTER AND METHODS OF USE AND ASSEMBLY

(71) Applicant: Regeneron Pharmaceuticals, Inc., Tarrytown, NY (US)

(72) Inventors: Alexei Goraltchouk, Cambridge, MA (US); Ian Hart, Nassau, NY (US); Mykhaylo Hrytsak, Hastings-on-Hudson, NY (US)

(73) Assignee: Regeneron Pharmaceuticals, Inc., Tarrytown, NY (US)

(21) Appl. No.: 15/017,200

(22) Filed: Feb. 5, 2016

Related U.S. Application Data
(60) Provisional application No. 62/113,157, filed on Feb. 6, 2015.

(51) Int. Cl. A61M 5/31; A61M 5/315

(52) U.S. Cl. CPC A61M 5/3135; A61M 5/31515; A61M 2005/3142; A61M 2207/00

(57) ABSTRACT

Syringe shoulder adapters, injection systems and methods for assembling and using the syringe shoulder adapter and injection system are disclosed. The syringe shoulder adapter including a housing with a first end and a second end opposite the first end. The housing including a cavity defined by an exterior surface between the first end and the second end and a first slot positioned near the second end of the housing and extending into the cavity. The injection system including a shoulder adapter, a syringe positioned within the shoulder adapter, and an injector coupled to the shoulder adapter. Methods of assembling and using the injection system are also disclosed.

### CROSS REFERENCE TO RELATED APPLICATION

This application claims priority benefit under 35 U.S.C. §119(e) to U.S. provisional application No. 62/113,157 filed Feb. 6, 2015, which is incorporated herein by reference in its entirety.

### FIELD OF THE INVENTION

The present invention relates generally to drug delivery systems for administering medication. More specifically, but not exclusively, the present invention concerns a shoulder adapter and injection system.

### BACKGROUND OF THE INVENTION

Currently many syringes, injectors and assisted injection systems apply a force on the flange of a syringe during injection. The force applied to the syringe flange may cause the flange to break or the syringe to shatter. If the syringe and/or flange breaks during the injection, the syringe pieces may cut the patient and/or the medical professional. In addition, the syringe may break before the injection is complete which may result in the medication or fluid flowing out of the syringe. If the syringe breaks such that the medication or fluid is lost, this may result in loss of expensive medications or difficulties in determining how much medication was administered to the patient. Thus, reduction or elimination of syringe flange breakage during the injection cycle is needed.

### SUMMARY OF THE INVENTION

Aspects of the present invention provide a syringe shoulder adapter and injection system. The present invention also provides methods for assembling and using the syringe shoulder adapter and injection system.

In one aspect provided herein is a syringe shoulder adapter including a housing with a first end and a second end opposite the first end. The housing including a cavity defined by an exterior surface between the first end and the second end and a first slot positioned near the second end of the housing and extending into the cavity.

In another aspect, provided herein is an injection system including a shoulder adapter, a syringe positioned within the shoulder adapter, and an injector coupled to the shoulder adapter.

In yet another aspect, provided herein is a method of assembling an injection system, the method includes obtaining a syringe shoulder adapter, a syringe, and an injector device. The method also includes inserting the syringe into a cavity in the shoulder adapter. The method further includes attaching the injector device to a coupling mechanism on a first end of the shoulder adapter.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a perspective view of a shoulder adapter from a first end, in accordance with an aspect of the present invention;

FIG. 2 is a top perspective view of the shoulder adapter of FIG. 1 from the first end, in accordance with an aspect of the present invention;

FIG. 3 is a top perspective view of the shoulder adapter of FIG. 1 from a second end, in accordance with an aspect of the present invention;

FIG. 4 is a top view of the shoulder adapter of FIG. 1, in accordance with an aspect of the present invention;

FIG. 5 is a side view of the shoulder adapter of FIG. 1, in accordance with an aspect of the present invention;

FIG. 6 is a bottom view of the shoulder adapter of FIG. 1, in accordance with an aspect of the present invention;

FIG. 7 is a first end view of the shoulder adapter of FIG. 1, in accordance with an aspect of the present invention;

FIG. 8 is a second end view of the shoulder adapter of FIG. 1, in accordance with an aspect of the present invention;

FIG. 9 is a perspective view of the housing of the shoulder adapter of FIG. 1, in accordance with an aspect of the present invention;

FIG. 10 is a perspective view of the retaining member of the shoulder adapter of FIG. 1, in accordance with an aspect of the present invention;

FIG. 11 is a perspective view of the deformable member of the shoulder adapter of FIG. 1, in accordance with an aspect of the present invention;

FIG. 12 is a perspective view of an exploded injection system including the shoulder adapter of FIG. 1, in accordance with an aspect of the present invention;

FIG. 13 is a perspective view of the assembled injection system, in accordance with an aspect of the present invention; and

FIG. 14 is a perspective view of another shoulder adapter, in accordance with an aspect of the present invention.

### DETAILED DESCRIPTION FOR CARRYING OUT THE INVENTION

Generally stated, disclosed herein is a syringe shoulder adapter and injection system. Further, methods of assembling and using the syringe shoulder adapter and injection system are discussed.

In this detailed description and the following claims, the words proximal, distal, anterior, posterior, medial, lateral, superior and inferior are defined by their standard usage for indicating a particular part of a device according to the relative disposition of the device with respect to a body or directional terms of reference. For example, “proximal” means the portion of a device nearest the point of attachment, while “distal” indicates the portion of the device farthest from the point of attachment. As for directional terms, “anterior” is a direction towards the front side of the device, “posterior” means a direction towards the back side of the device, “medial” means towards the midline of the device, “lateral” is a direction towards the sides or away from the midline of the device, “superior” means a direction above and “inferior” means a direction below another object or structure.

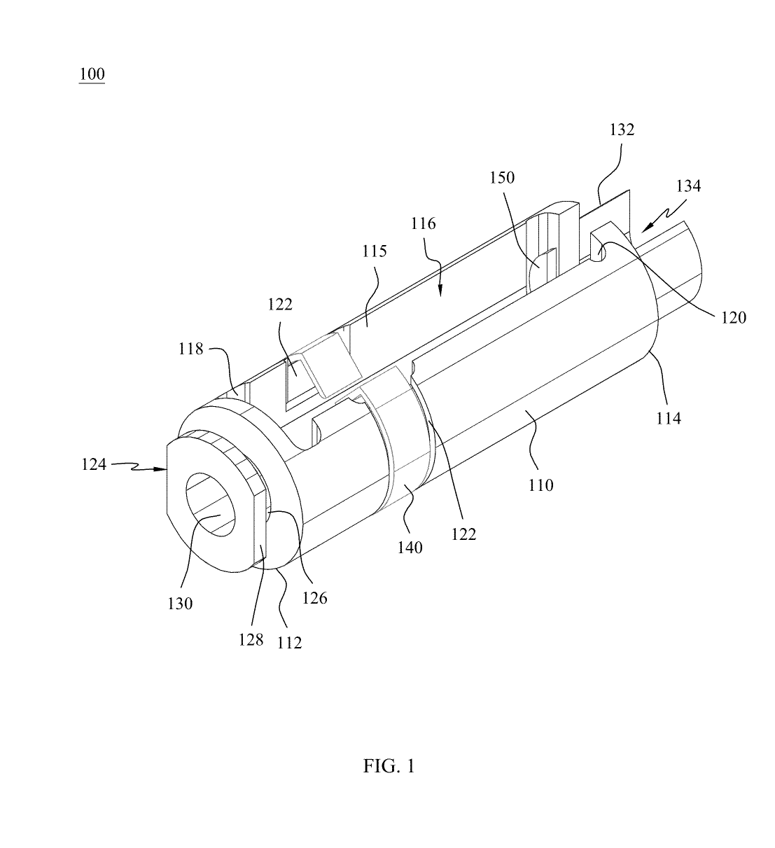

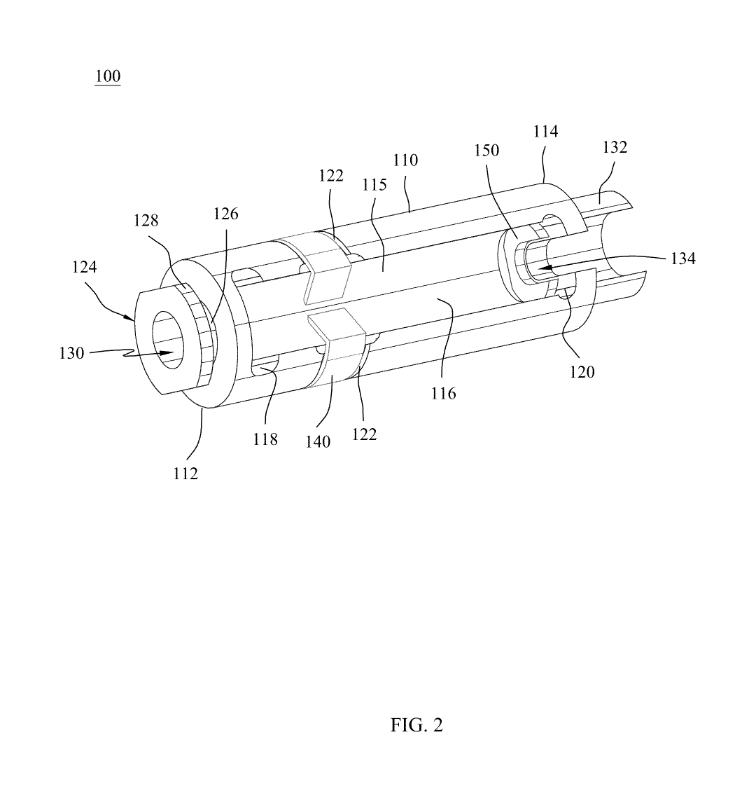

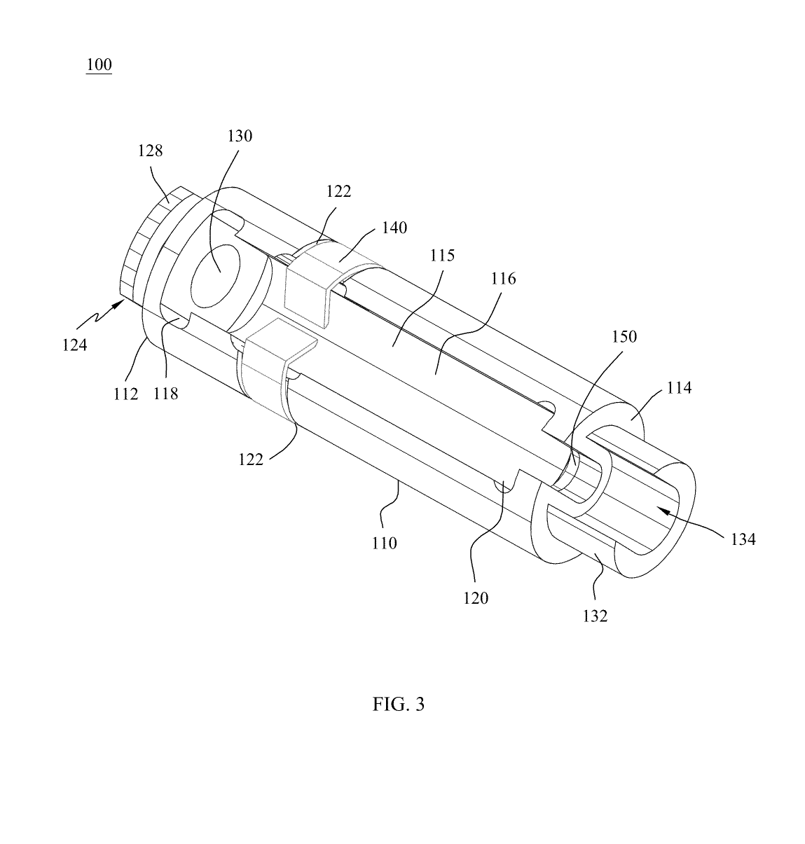

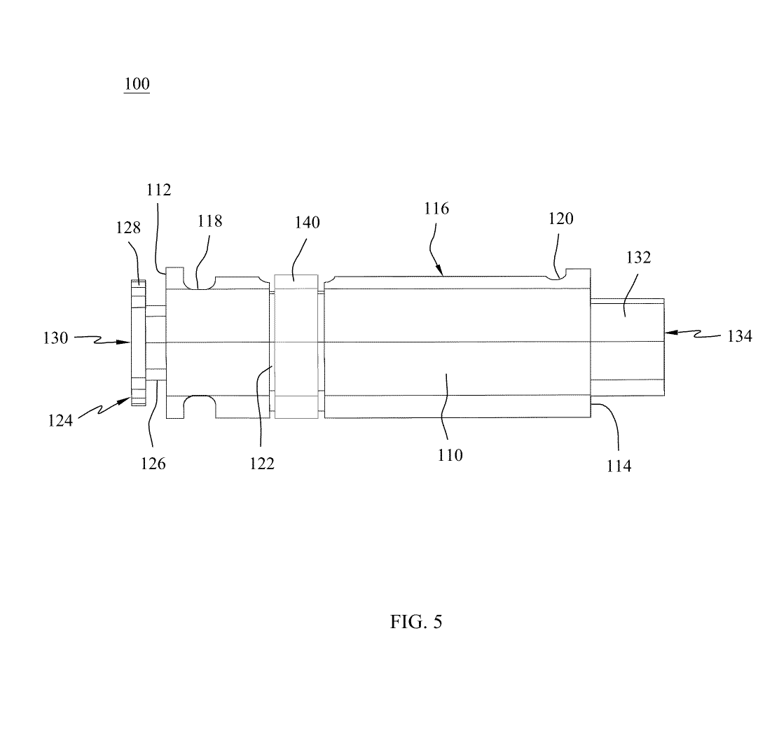

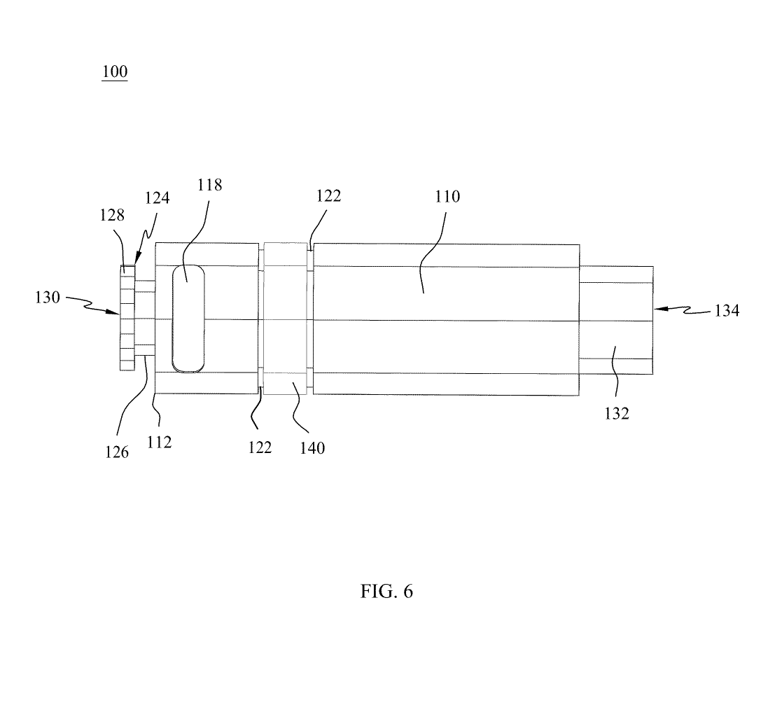

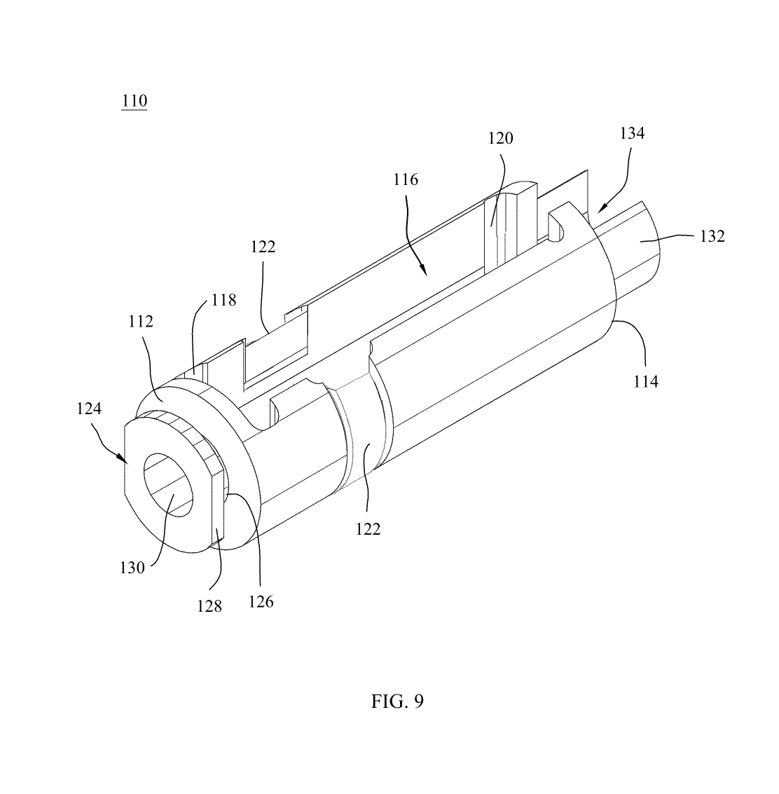

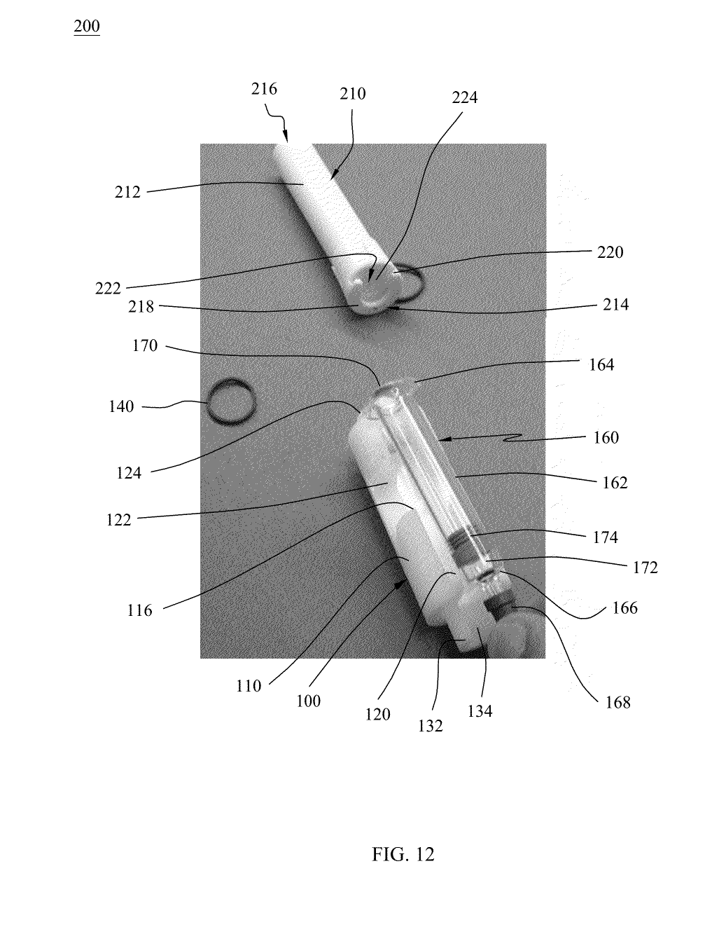

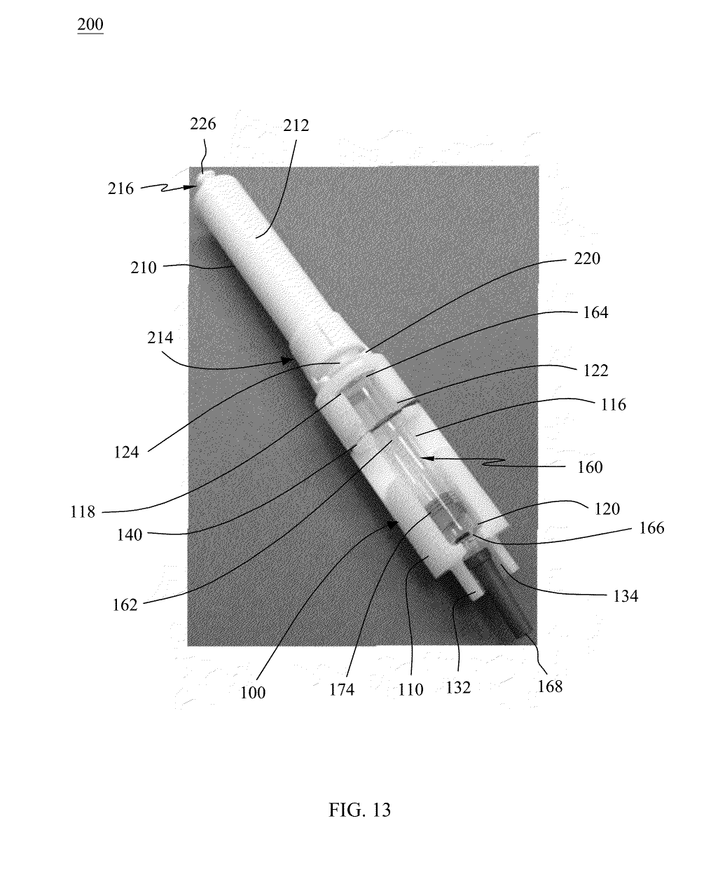

Referring to the drawings, wherein like reference numerals are used to indicate like or analogous components throughout the several views, and with particular reference to FIGS. 1-11, there is illustrated a syringe shoulder adapter 100. The shoulder adapter 100 includes a housing 110, a retaining member 140, and a deformable member 150. The housing 110 may include a first end 112 and a second end 114 with a sidewall 115 extending between the first end 112 and the second end 114. The housing 110 may also include a cavity 116 extending into the housing 110 from an exterior surface. The cavity 116 may extend from the first end 112 to the second end 114. The cavity 116 may be sized to receive a medication storage and delivery device, such as a syringe 160 shown in FIGS. 12-13 or a cartridge (not shown). When the syringe 160 is inserted into the cavity 116, the cavity 116 creates a window to view the syringe 160 during the injection cycle. The cavity 116 may include a first slot 118 near the first end 112 of the housing 110 and a second slot 120 near the second end 114 of the housing 110. The second slot 120 may receive the deformable member 150 and a portion of the syringe 160. The deformable member 150 may act, for example, to cushion the interface between the syringe 160 and the second end 114 of the housing 110. Although not shown, it is also contemplated that a second deformable member 150 may be positioned in the first slot 118. The housing 110 may also include a groove 122 extending around the exterior surface of the sidewall and into the cavity 116. The groove 122 may be sized to receive the retaining member 140. Alternatively, the retaining member 140 may be, for example, integral with the housing 110 and positioned where the groove 122 is shown.

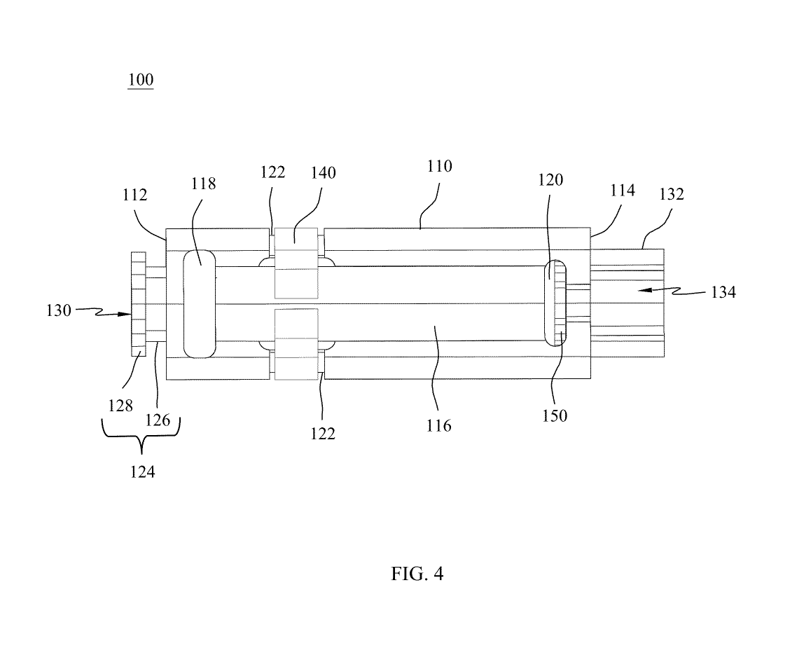

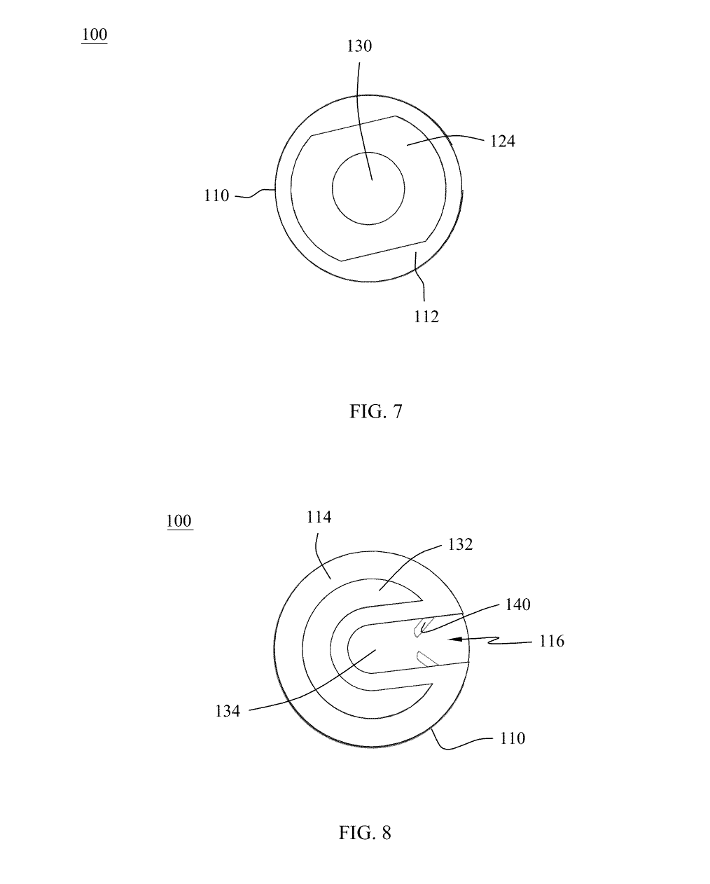

With continued reference to FIGS. 1-9, the housing 110 may include a coupling mechanism 124 extending away from the first end 112. The coupling mechanism may include a neck 126 and a lip 128, as best shown in FIGS. 4-6. The neck 126 may be, for example, generally cylindrical and extend out from the first end 112 of the housing 110. The lip 128 may extend out relatively perpendicular from the neck 126. The lip 128 of the coupling mechanism 124 may have, for example, two planar sides and two rounded or curved sides with the two planar sides positioned opposite each other and the two rounded sides positioned opposite each other, as shown in FIG. 7. The lip 128 may be shaped to function, for example, in positioning and/or locking the shoulder adapter 100 to the injection mechanism. The housing 110 may also include an opening 130 extending through the coupling mechanism 124 and into the cavity 116 at the first end 112. The opening 130 may be positioned, for example, centered in the first end 112 of the housing 110, such that the opening 130 extends into the center of the cavity 116. The housing 110 may further include a projection 132 extending away from the second end 114. In addition, the housing 110 may include a channel 134 recessed in the projection 132 and extending through the second end 114 of the housing 110 into the cavity 116. The projection 132, as shown in FIGS. 1-6 and 8-9, may be sized, for example, to act as a needle length gauge to limit the depth of needle penetration into the skin. Thus, the adapter 100 may come with varying length projections 132 to ensure the injection mechanism is inserted to the proper depth for injection of a given medication into the patient. It is also contemplated that the projection 132 may, for example, cover the entire injection mechanism to allow for topical application of medications.

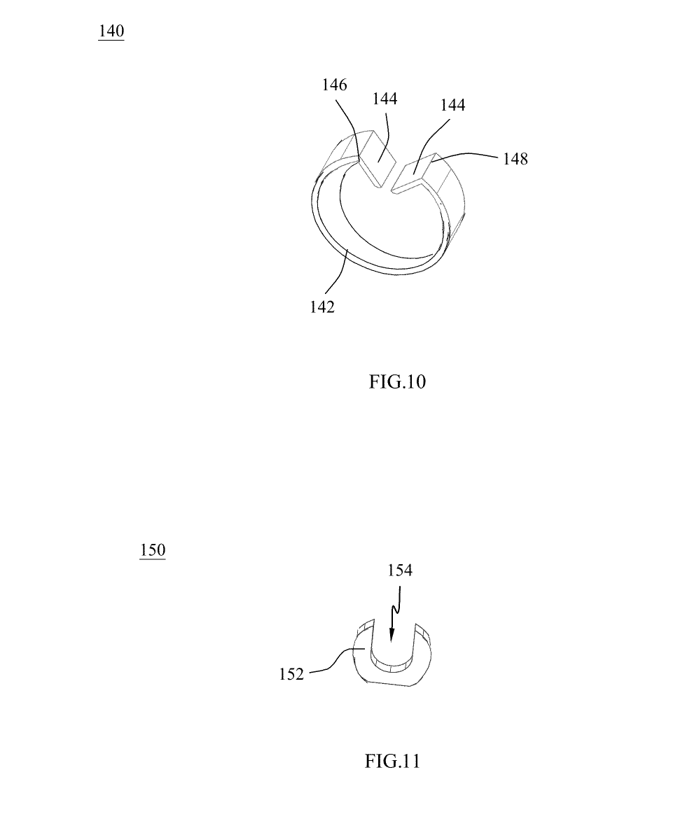

Referring now to FIG. 10, an example retaining member 140 is shown. The retaining member 140 may be, for example, a unitary piece or have at least two pieces coupled together. The retaining member 140, as illustrated in FIGS. 1-6 and 10, may be a spring clip and include a cylindrical body portion 142 and at least two engagement members 144. The cylindrical body portion 142 may include a first end 146 and a second end 148. The cylindrical body portion 142 may be sized to fit around the housing 110 in the groove 122. The cylindrical body portion 142 may also include a gap between the first end 146 and the second end 148 and the gap may be, for example, the width of the cavity 116. A first engagement member 144 may extend away from the first end 146 into the gap and a second engagement member 144 may extend away from the second end 148 into the gap. When the retaining member 140 is inserted onto the housing 110, the at least two engagement members 144 may extend into the cavity 116 to secure a syringe in the cavity 116 of the housing 110. Alternative retaining members 140 are also contemplated and may include, for example, a spring clip, at least one elastomeric element, an elastic member, a rubber band, soft rubber bumpers, elastomeric extrusions of low durometer, and the like. As shown in FIGS. 12 & 13, the retaining member 140 may be, for example, a rubber band like structure that may be positioned in a groove 122, which is sized to receive the rubber band. If bumpers are used they may be positioned, for example, at the top of the cavity 116 and will deform to allow a syringe to be inserted into the cavity 116 and assist in retaining the syringe in the cavity during the injection cycle. It is also contemplated that the retaining member 140 may be, for example, integral to the housing 110 and include an opposing first engagement member 144 and second engagement member 144 which each extend out from the housing 110 into the cavity 116.

Referring now to FIG. 11, an example deformable member 150 is shown. The deformable member 150 may be, for example, a gasket, spacer, or the like. The deformable member 150 may be fabricated from, for example, a thermoplastic, elastomer, or thermoset elastomer, such as, silicone, polyvinylidene fluoride (PVDF), elvax, butyl rubber, natural rubber, nitride rubber, fluoro-elastomer, vinyl, nylon, polyurethane elastomer, halo-butyl rubber, neoprene, and the like. The deformable member 150 may conform to the shape of a syringe or cartridge inserted into the housing 110 to spread the compressive load across the deformable member 150. The deformable member 150 may include a body 152 and a channel 154. The body 152 may be, for example, generally cylindrical and sized to fit in the second slot 120 of the housing 110. The channel 154 may be sized to match the size of the channel 134 in the housing 110. The deformable member 150 may be removable or placed permanently in the second slot 120 of the housing 110. If the deformable member 150 is removable, the deformable member 150 may optionally be attached to the second slot 120 at the second end 114 by, for example, by an adhesive material.

An example injection system 200 is shown in FIGS. 12-13. The injection system 200 may include the syringe shoulder adapter 100, a syringe 160, and an injector device 210. The syringe 160 may include a body 162 with a flange 164 at a first end and a shoulder 166 at a second end. An injection mechanism (not shown) may extend out from the second end of the body 162 and may optionally include a cover 168 over the injection mechanism. The terms “cover,” “cap,” “needle cover” and “shield” may be used interchangeably herein as they each refer to a structure used to maintain a sterile field and protect the patient and medical professional from accidentally being stuck by the injection mechanism. The injection mechanism may be, for example, a needle, microneedle, cannula, or the like for a subcutaneous injection or a tube, dispensing needle, or the like for topical application to the skin, a patch, or the like. The first end of the body 162 may also include an opening 170 for receiving a fluid or medication 172 and a piston head 174 for injection into a patient. The terms “piston head,” “plunger” and “plunger head” may be used interchangeably herein as they each refer to the same structure used for injecting a medication into the patient. In the embodiments shown in FIGS. 12-13, the piston head 174 is illustrated in a nearly complete injection position. In the syringe 160, that is full of medication 172 which has not yet been administered, the piston head 174 will be positioned near the first end closer to the flange 164 of the syringe 160. The cavity 116 of the adapter 100 may be sized to receive the syringe 160.

The injector device 210 may include a housing 212 with an adapter end 214 and an activation end 216, as shown in FIGS. 12 and 13. The adapter end 214 may include a coupling portion 218 with a recess 220 and an opening 222. The recess 220 may extend into the housing 212 from an exterior surface in a direction perpendicular to the longitudinal axis of the housing 212. The recess 220 may be sized to receive the lip 128 of the coupling mechanism 124 of the housing 110. The opening 222 may extend from the second end into the housing 212 in a direction along the longitudinal axis of the housing 212. The opening 222 may be sized to receive the neck 126 of the coupling mechanism 124 of the housing 110. The coupling portion 218 may be, for example, generally horseshoe or C shaped. The adapter end 214 may also include a plunger rod 224 recessed within the housing 212. The plunger rod 224 may be positioned such that upon release from the housing 212 the plunger rod 224 extends through the opening 222 to contact the piston head 174 in the syringe 160 to inject the medication or fluid 172. As shown in FIG. 13, the activation end 216 may include a deployment means 226 allowing for the deployment or the pushing of the plunger rod 224 out of the housing 212. The deployment means 226 may be, for example, a compressed air or gas line for an assisted injection system or alternatively, a manual injection mechanism coupled to the plunger rod 224 or an opposite end of the plunger rod 224 may be used for manual injection system. The housing 110 may also include, for example, a door or moveable portion (not shown) at the first end 112 which may be opened to access the opening 130 and a portion of cavity 116. The door or moveable portion (not shown) of housing 110 would allow for the syringe shoulder adapter 100 to be removed from an injection system 200 without damage by providing a passage for removal of the plunger rod 224 after injection. By allowing for the removal of the plunger rod 224 from the adapter 100 after injection, the adapter 100 may be reusable.

The injection system 200 may be assembled by first obtaining a syringe shoulder adapter 100, a syringe 160 filled with the desired fluid or medication 172 for injection, and an injector device 210. The syringe shoulder adapter 100 should be selected to correspond to the size of the syringe 160 being used. If the deformable member 150 is not permanent, then the deformable member 150 may be inserted into the second slot 120 and optionally, attached to the housing 110 using, for example, an adhesive material. The adhesive material may be, for example, temporary or permanent in nature. Once the deformable member 150 is positioned within the second slot 120, the syringe 160 may be inserted into the housing 110.

To insert the syringe 160 into the housing 110, the syringe flange 164 may be aligned with the first slot 118 of the syringe shoulder adapter 100 and the syringe cover 168 may be aligned with the channel 134. Next, a force may be applied to the syringe 160 to insert the syringe 160 into the cavity 116. Once the syringe 160 is positioned in the cavity 116, a retaining member 140, for example, the spring clip of FIG. 10 or the elastic member of FIGS. 12-13, may optionally be inserted into the groove 122 to secure the syringe 160 into the housing 110. The size of the groove 122 may correspond to the size of the retaining member 140, such that the groove 122 for receiving the spring clip 140 of FIG. 10 will be larger than the groove 122 for receiving the elastic member 140 of FIGS. 12-13. If a spring clip 140 is used, the spring clip 140 may be pressed into the housing 110 at the groove 122. Alternatively, if an elastic member 140 is used, the elastic member 140 may be inserted over the first or second end 112, 114 of the housing 110 and moved into position within the groove 122.

Next, the injector device 210 may be attached to the coupling mechanism 124 of the syringe shoulder adapter 100, as shown in FIG. 13. The injector device 210 may be attached by aligning the lip 128 of the adapter coupling mechanism 124 with the recess 220 in the adapter end 214 of the injector 210. Once the lip 128 is aligned with the recess 220, a force may be applied by the user to insert the lip 128 into the recess 220 and the neck 126 into the opening 222. The recess 220 may be, for example, sized to secure the coupling mechanism 124 to the injector device 210 such that a force is required to release the coupling mechanism 124 from the recess 220. Finally, if necessary, an actuation mechanism may be attached to the deployment means 226 of the injector device 210.

Once assembled, the injection system 200 may be used to deliver a fluid or medication 170 to a patient. To initiate the injection cycle, the cap or cover 168 would be removed from the syringe 160 to expose the needle (not shown). Next, the needle may be inserted into the patient for injection or positioned over the area of application for a topical application. Once the needle (or patch) is in the desired position, the deployment means 226 may be activated. As the deployment means 226 is activated, a force is applied to the plunger rod 224 to move the plunger rod out of the housing 212 of the injector device 210. As the plunger rod 224 moves it passes through the opening 222 in the injector device 210 and into the opening 130 in the syringe shoulder adapter 100. The opening 130 acts to align the plunger rod 224 with the inner diameter of the syringe body 162 to avoid uneven pressure and point loading on the flange 164. The plunger rod 224 continues through the opening 130 and into the opening 170 in the syringe 160. As the plunger rod 224 passes into the syringe 160, the plunger rod 224 contacts the piston head 174. The force of the plunger rod 224 on the piston head 174 causes the piston head 174 to begin to move toward the shoulder 166. As force is applied to the plunger rod 224 and in turn the piston head 174, the first slot 118 provides support to the flange 164 to prevent breakage of the flange 164. In addition, the syringe shoulder adapter 100 transfers the force being applied to the plunger rod 224 and piston head 174 to the syringe shoulder 166 which contacts the deformable member 150 and assists in minimizing the resultant load on the flange 164. As the piston head 174 moves, the medication or fluid 172 is forced out of the syringe 160 and through the needle (not shown) into the patient. The deployment means 226 will continue to apply a force on the plunger rod 224 until the desired amount of fluid or medication 172 is administered to the patient. As the injection cycle occurs, the cavity 116 provides a window to view the syringe 160 to allow the medical professional or user to confirm that the desired amount of fluid or medication 172 injected into the patient. Once the desired amount of medication or fluid 172 is administered, the needle may be removed from the patient and the cap 168 may be replaced to cover the needle.

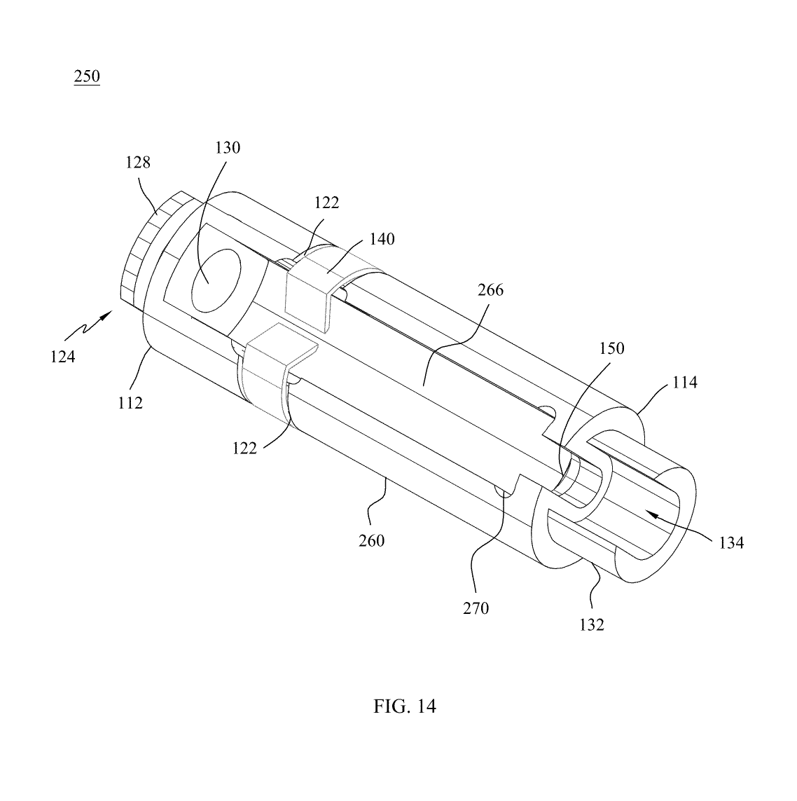

Referring now to FIG. 14, another shoulder adapter 250 is shown. The shoulder adapter 250 is shaped to receive, for example, a cartridge or other tube-like medication storage device without a flange. The shoulder adapter 250 may include a housing 260, a retaining member 140, and a deformable member 150. The housing 260 may be of the type described above with reference to housing 110 without the slot 118 near the first end 112. For brevity sake, the housing 260 will not be described again here in detail. The housing 260 may also include a cavity 266 extending into the housing 110 from an exterior surface. The cavity 266 may extend from the first end 112 to the second end 114. The cavity 266 may be sized to receive a medication storage and delivery device, such as a cartridge without a flange. When the cartridge (not shown) is inserted into the cavity 266, the cavity 266 creates a window to view the cartridge during an injection. The cavity 266 may also include a first slot 270 near the second end 114 of the housing 260. The first slot 270 may be of the type described above with reference to second slot 120 which will not be described again here for brevity sake. The housing 260 may also include groove 122, coupling mechanism 124, lip 128, opening 130, projection 132, and channel 134 as described above with reference to housing 110 which will not be described again here for brevity sake.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. A shoulder adapter, comprising:
a housing with a first end and a second end opposite the first end, the housing comprising:
a cavity defined by an exterior surface between the first end and the second end; and
a first slot positioned near the second end of the housing and extending into the cavity.

2. The shoulder adapter of claim 1, further comprising:
a retaining member removably coupled to the housing.

3. The shoulder adapter of claim 2, wherein the housing further comprises:
a groove positioned in the exterior surface of the housing for receiving the retaining member.

4. The shoulder adapter of claim 1, wherein the housing further comprises:
a coupling mechanism extending away from the first end; and
a projection extending away from the second end.

5. The shoulder adapter of claim 4, wherein the coupling mechanism comprises:
a neck; and
a lip, wherein the lip extends from the neck.

6. The shoulder adapter of claim 4, wherein the housing further comprises:
a channel extending from the cavity through the second end of the housing and the projection.

7. The shoulder adapter of claim 1, further comprising:
a deformable member positioned within the second slot.

8. The shoulder adapter of claim 2, wherein the retaining member is selected from the group consisting of a spring clip and an elastic member.

9. An injection system, comprising:
a shoulder adapter;
a syringe positioned within the shoulder adapter; and
an injector coupled to the shoulder adapter.

10. The injection system of claim 9, wherein the shoulder adapter comprises:
a housing with a first end, a second end, and a side wall extending between the first end and the second end, wherein the housing comprises:
a central cavity extending from the first end to the second end;
a first slot positioned proximate the second end of the housing and extending into the cavity;
a coupling mechanism extending away from the first end; and
a projection extending away from the second end.

11. The injection system of claim 10, wherein the shoulder adapter further comprises:
a retaining member positioned on the housing and overlapping at least a portion of the cavity, wherein the retaining member engages the syringe; and
a deformable member positioned within the first slot.

12. The injection system of claim 11, wherein the housing further comprises:
a groove disposed in the exterior surface of the housing for receiving the retaining member.

13. The injection system of claim 10, wherein the syringe comprises:
a body with a first end, a second end, and an opening extending into the body from the first end toward the second end;
a flange extending away from the first end of the body in a direction perpendicular to the longitudinal axis of the body;
a shoulder at the second end of the body;
an injection mechanism extending from the shoulder and into the opening of the body; and
a piston head positioned within the opening of the body.

14. The injection system of claim 13, wherein the housing further comprises:
a second slot positioned proximate the first end of the housing and extending into the cavity; and
the flange of the syringe is positioned in the second slot of the shoulder adapter, the shoulder of the syringe is positioned in the first slot of the shoulder adapter, and the injection mechanism extends from the shoulder adapter through the projection.

15. The injection system of claim 10, wherein the coupling mechanism comprises:
a neck extending away from the first end of the housing; and
a lip extending from the neck in a perpendicular direction.

16. The injection system of claim 15, wherein the injector comprises:
a housing with an adapter end, an activation end opposite the adapter end, and a plunger rod positioned inside the housing;
wherein the adapter end comprises:
a coupling portion with a recess for receiving the lip of the coupling mechanism of the shoulder adapter and an opening extending from the exterior surface of the housing into the recess for receiving the neck of the shoulder adapter;
wherein the activation end comprises:
a deployment means for activating the plunger rod.

17. A method of assembling an injection system, comprising:
obtaining a shoulder adapter, a syringe, and an injector device;
inserting the syringe into a cavity in the shoulder adapter; and
attaching the injector device to a coupling mechanism on a first end of the shoulder adapter.

18. The method of claim 17, wherein the shoulder adapter comprises:
a first end;
a second end opposite the first end;
a side wall extending between the first end and the second end;
the cavity extending along a longitudinal axis of a housing;
a first slot positioned near the second end of the housing and connected to the cavity;
a coupling mechanism including a neck extending from the first end and a lip extending from the neck in a perpendicular direction; and
a projection extending out from the second end and including a channel;
wherein the syringe comprises:
a body with a first end, a second end, and an opening extending into the body from the first end towards the second end;
a shoulder at the second end of the body;
an injection mechanism extending from the shoulder and into the opening of the body; and
a piston head positioned within the opening of the body; and
wherein the injector device comprises:
a housing with an adapter end, an activation end opposite the adapter end, and a plunger rod positioned inside the housing;
wherein the adapter end comprises:
a coupling portion with a recess and an opening extending from the exterior surface of the housing into the recess;
wherein the activation end comprises:
a deployment means coupled to the plunger rod.

19. The method of claim 18, wherein the shoulder adapter further comprises a second slot positioned near the first end of the housing and connected to the cavity, the syringe further comprises a flange extending out from the first end of the body in a direction perpendicular to the longitudinal axis of the body, and inserting the syringe into a cavity in the shoulder adapter comprises:
aligning the flange of the syringe with the first slot of the shoulder adapter;
aligning the shoulder of the syringe with the second slot of the shoulder adapter;
aligning the injection mechanism of the syringe with the channel of the projection of the shoulder adapter; and
applying a force to the syringe to move it into the cavity of the shoulder adapter.

20. The method of claim 18, wherein attaching the injector to a coupling mechanism on a first end of the shoulder adapter comprises:
aligning the lip of the shoulder adapter with the recess of the injector and the neck of the shoulder adapter with the opening of the injector; and
applying a force to move the lip into the recess and neck into the opening.
